(19) United States
(12) Patent Application Publication
(10) Pub. No.: US 2015/0057565 A1
(43) Pub. Date: Feb. 26, 2015

(54) APPARATUS AND METHOD FOR OVARIAN CANCER SCREENING

(71) Applicant: MKT Enterprises, LLC, South Windsor, CT (US)

(72) Inventors: Thomas G. Mazzoli, JR., Somers, CT (US); Maria Lynne Ellis, South Windsor, CT (US); Kimberly Ann McDonald, South Windsor, CT (US); David Freed, Durham, NC (US)

(21) Appl. No.: 14/464,809

(22) Filed: Aug. 21, 2014

Related U.S. Application Data
(60) Provisional application No. 61/868,298, filed on Aug. 21, 2013.

(51) Int. Cl. A61B 10/02; A61B 10/04; A61M 25/10

(52) U.S. Cl. CPC A61B 10/0291; A61M 25/1002; A61B 10/04; A61B 10/0266; A61M 2025/105; A61M 2210/1082; A61M 2210/1425; A61M 2210/1408
USPC 600/564; 604/506; 600/569

(57) ABSTRACT

An apparatus and method are provided for sampling the distal tube, fimbria and/or ovary, includes advancing a device into the peritoneal cavity and sampling material on or adjacent to the distal tube, fimbria or ovary.

### CROSS REFERENCE TO RELATED APPLICATIONS

This application claims the benefit of U.S. Provisional Application No. 61/868,298 filed Aug. 21, 2013, the disclosure of which is incorporated by reference herein in its entirety.

### BACKGROUND

The present disclosure relates to an apparatus and method for ovarian cancer screening, particularly to an apparatus and method for sampling the distal tube, fimbria and/or ovary via catheter accessing the peritoneal cavity via the cul de sac.

Traditional approaches for ovarian cancer screening have either been though the cervix and into the fallopian tubes or via ovarian removal. For example, U.S. Patent Publication No. 2012/0315662 to Linnemeier describes detection of precancerous changes in the fallopian tubes. The described mechanism includes insertion of a catheter through the cervix and into the fallopian tubes, with a cervical brush sampling cells from within the fallopian tubes. The methodology is described as flowing from hysteroscopy, and techniques used therein, which utilizes a catheter that is advanced into the fallopian tube. The reference further indicates that using a transcervical approach, one Essure micro-insert is placed in the proximal portion of each fallopian tube lumen.

U.S. Pat. No. 6,984,498 to Adair describes a method of cancer screening using non-invasive or minimally invasive techniques. This reference describes cell retrieval alternately through non-invasive (exfoliation via approach through the cervix with a cytological brush) and minimally invasive means (as a peritoneal cavity approach via peritoneal lavage catheter).

In general, previous screening methods include measurement of CA-125 serum elevations, ultrasound, peritoneal tap and imprint cytology. The CA-125 antigen can be elevated in most, but not all, women with ovarian cancer. Unfortunately, it is non-specific. In fact, most women with a serum elevation of CA-125 will not have ovarian cancer. Many benign conditions are associated with serum elevations. In fact, normal physiologic states, such as active menses, can cause elevation of this antigen.

With regard to ultrasound, there are grading systems that take into account features, such as the presence of cysts, the complexity of cysts and the presence of ascites fluid. This is a good tool, but not specific enough to be used as a primary screening tool.

With a peritoneal tap, even in cases where a patient is ultimately diagnosed with ovarian cancer, a sample of the ascites fluid contains cancer cells less than half of the time.

With regard to imprint cytology, pathologists have demonstrated that a smear from the ovarian surface is useful for identifying ovarian cancer. However, this procedure requires abdominal exploration under general anesthesia and removal of the pelvic mass.

Currently know procedures that access the cul de sac include culdoscopy, which involves generally placing a scope through though the cul de sac, and culdocentisis, which involves sampling of pelvic fluid in the cul de sac. Rigid culdoscopy has been reported in the literature for most of the last century. It was essentially abandoned with the development of laparoscopy. It has also recently been proposed as a helpful addition during operative laparoscopy.

Culdocentesis, which involves sampling of pelvic fluid, has not been shown to be accurate in detecting ovarian malignancy even when the disease is known to be present.

Previous culdoscopic approaches to identifying pelvic disease have focused mainly on conditions affecting female fertility. Recently this vaginal approach has also been suggested as an adjunct to other operative procedures (e.g., laparoscopic procedures). Rarely authors have suggested sampling pelvic fluid or removing portions of Fallopian tube or ovary for biopsy (with or without imprint cytology, as has been described above, to assist in surgical decision making at the time of ovarian removal).

What is needed in the art is an improvement to ovarian cancer screening and an alternative to cervical and laparoscopic approaches for ovarian cancer screening.

### BRIEF SUMMARY OF THE INVENTION

The above discussed and other drawbacks and deficiencies are overcome or alleviated by the present apparatus and method for sampling the distal tube, fimbria and/or ovary in the peritoneal cavity via a medical device. Exemplary embodiments include a catheter accessing the peritoneal cavity through the cul de sac, which includes piercing the cul de sac through the vaginal wall, advancing a catheter through the cul de sac and inflating a balloon operatively associated with the catheter to lift the uterus, continuing advance the catheter into the peritoneal cavity to a position proximate to a distal tube, fimbria or ovary and sampling material on or adjacent to the distal tube, fimbria or ovary.

In exemplary embodiments, sampling is performed with a brush that extends from and retracts into said catheter. In other exemplary embodiments, sampling may be via aspirating of fluid. In other exemplary embodiments, sampling may be by one or more of a retractable brush, a stationary brush, a needle, a cutting device (e.g., scissors or grader), a suction device, or a powered removal device (e.g., laser cutting device, powered scissors, etc.). Additionally, the catheter may additionally sample on a contralateral side.

In other exemplary embodiments, the catheter comprises a scope configured to visualize a target, which catheter may also include a steerable distal portion to assist in positioning the distal end of the scope.

As we have mentioned above, the balloon is configured to balloon is configured to lift the uterus. In further exemplary embodiments, the balloon is configured with two wings complementary to the natural shape of the uterus.

In additional exemplary embodiments, access may be facilitated with the use of a vaginal speculum and a tenaculum to expose the access point. Access may also be provided by a sheathed needle, followed by a trocar to dilate the sheath.

The present apparatus and method advantageously takes advantage of a culdoscopic approach in combination with sampling techniques from within the peritoneal cavity to identify ovarian cancer or precursor cells. The known art only uses imprint cytology, which is a smear of the ovary after ovarian removal (e.g., in the pathology lab), to assist in surgical decision making at the time of ovarian removal.

The presently described technique can also be provided as a brief screen of women concurrent with a colonoscopy. Women at risk for ovarian cancer are in the same age group as women who are traditional candidates for colonoscopy screening. In addition, the literature suggests a low complication rate for culdoscopy, the most common complication being bowel perforation; and women who present for colon cancer screening have already consented to a procedure with the same risk profile. Colonoscopy suites typically function with high volume and quick room turnover, and the present method could reasonably be performed within 30 minutes.

Patients identified with cancer would be better able to be counseled preoperatively and indeed may opt to schedule their procedure at a different time or facility so that a gynecologic-oncologist would be readily available. Women who have benign results may also be able to observe pelvic cysts. Additionally, in the case of women with genetic risk for cancer, reassuring results may assist in the decision regarding timing of risk reduction surgery (e.g., many women want to complete their families or avoid menopausal symptoms until later ages).

Finally, the present apparatus and method provide for direct sampling from the ovary/fallopian tube complex without removal of potentially normal organs.

The above-discussed and other features and advantages of the present invention will be appreciated and understood by those skilled in the art from the following detailed description and drawings.

### BRIEF DESCRIPTION OF THE DRAWINGS

Referring to the FIGURES wherein like elements are numbered alike in the several FIGURES:

FIG. 1A illustrates a plan view of a catheter apparatus in accordance with exemplary embodiments of the present invention;

FIG. 1B illustrates a cross sectional view taken across 1-1 of FIG. 1A of a catheter apparatus in accordance with exemplary embodiments of the present invention;

FIG. 2 illustrates a cross sectional view of a catheter add-on placed on a scope in accordance with exemplary embodiments of the present invention;

FIG. 3A illustrates a plan view of an un-inflated balloon in accordance with exemplary embodiments of the present invention;

FIG. 3B illustrates a plan view of an inflated balloon in accordance with exemplary embodiments of the present invention;

FIG. 4 illustrates a perspective view of patient in a genupectoral position in accordance with exemplary embodiments of the present invention;

FIG. 5 illustrates use of a speculum/retractor to provide access to the vagina in accordance with exemplary embodiments of the present invention;

FIG. 6 illustrates treatment of the vagina with Betadyne in accordance with exemplary embodiments of the present invention;

FIG. 7 illustrates use of a tenaculum to expose the USLs in accordance with exemplary embodiments of the present invention;

FIG. 8 illustrates use of a sheathed needle to access the cul de sac in accordance with exemplary embodiments of the present invention;

FIG. 9 illustrates dilation of the sheath of FIG. 8 with a trocar in accordance with exemplary embodiments of the present invention;

FIG. 10 illustrates advancement of the catheter and inflation of a balloon in accordance with exemplary embodiments of the present invention;

FIG. 11 illustrates sampling of the distal tube/proximal ovary in accordance with exemplary embodiments of the present invention;

FIG. 12 illustrates retraction of the sample in accordance with exemplary embodiments of the present invention;

FIG. 13 illustrates sampling on a contralateral side in accordance with exemplary embodiments of the present invention;

FIG. 14 illustrates retraction of the sample from the contralateral side in accordance with exemplary embodiments of the present invention;

FIG. 15 illustrates withdrawal of the instruments in accordance with exemplary embodiments of the present invention;

FIG. 16 illustrates a kit in accordance with exemplary embodiments of the present invention;

FIG. 17 illustrates a plan view of an exemplary brush;

FIG. 18 illustrates a side elevation view of an exemplary clickable handle;

FIG. 19 illustrates a cross sectional view of an exemplary brush inside a lumen with a ball to block fluid;

FIG. 20 a illustrates an exemplary uteroscope with sampling device and balloon;

FIG. 21 illustrates a closer, perspective view of an exemplary lumen for a sampling device;

FIG. 22 illustrates an exemplary device, including inflation lumen, a stopcock, a scope lumen and a balloon;

FIG. 23 further illustrates a plan view of an exemplary outer sheath, a flared sheath and a brush; and

FIG. 24 illustrates a side elevation view of an exemplary handle assembly and a Luer for fluid connection.

### DETAILED DESCRIPTION OF THE INVENTION

As was noted above, the present disclosure provides an apparatus and method for sampling the distal tube, fimbria and/or ovary via a medical device. Exemplary embodiments provide a catheter accessing the peritoneal cavity through the cul de sac, which includes piercing the cul de sac through the vaginal wall, advancing a catheter through the cul de sac and inflating a balloon operatively associated with the catheter to lift the uterus, continuing advance the catheter into the peritoneal cavity to a position proximate to a distal tube, fimbria or ovary and sampling material on or adjacent to the distal tube, fimbria or ovary.

In exemplary embodiments, sampling is performed with a brush that extends from and retracts into said catheter. In other exemplary embodiments, sampling may be via aspirating of fluid. In other exemplary embodiments, sampling may be by one or more of a retractable brush, a stationary brush, a needle, a cutting device (e.g., scissors or grader), a suction device, or a powered removal device (e.g., laser cutting device, powered scissors, etc.). Additionally, the catheter may additionally sample on a contralateral side.

In addition to sampling, the device may be configured to deposit materials on or near the distal tube, fimbria or ovary.

In other exemplary embodiments, the catheter comprises a scope configured to visualize a target, which catheter may also include a steerable distal portion to assist in positioning the distal end of the scope.

As we have mentioned above, the balloon is configured to balloon is configured to lift the uterus. In further exemplary embodiments, the balloon is configured with two wings complementary to the natural shape of the uterus.

In additional exemplary embodiments, access may be facilitated with the use of a vaginal speculum and a tenaculum to expose the access point. Access may also be provided by a sheathed needle, followed by a trocar to dilate the sheath.

An exemplary apparatus and procedure will now be described with reference to the various FIGURES. While the FIGURES illustrate a particular exemplary apparatus and procedure, it should be understood that the invention is not limited thereto.

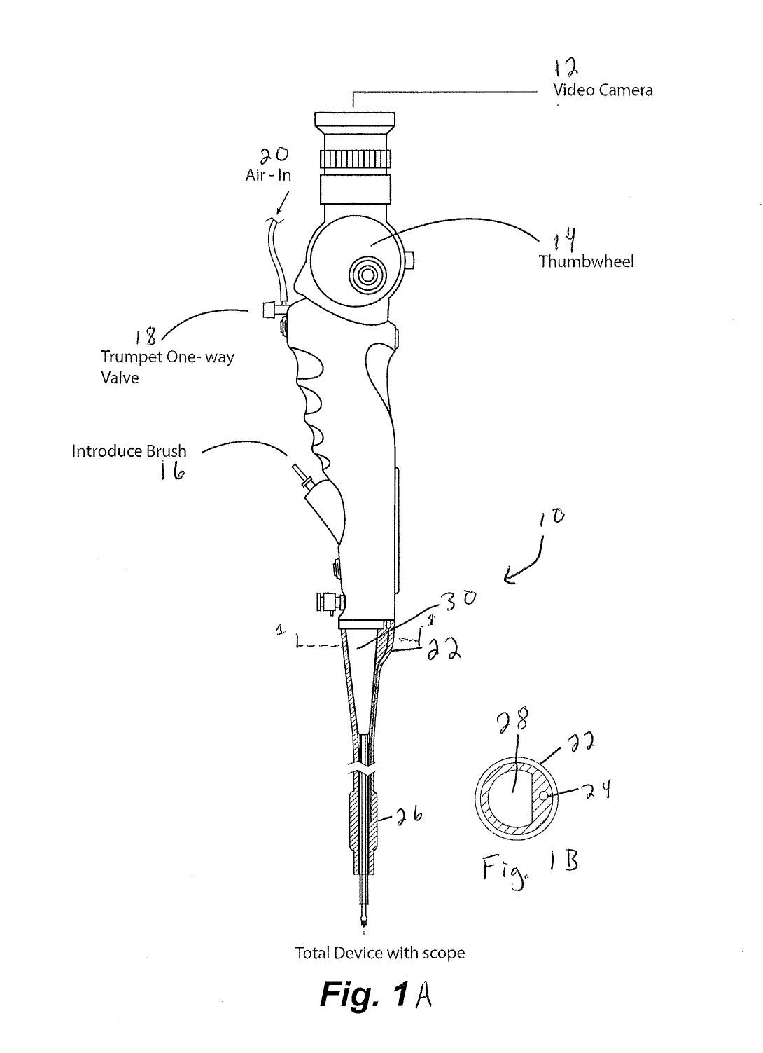

Referring now to FIG. 1A, an exemplary catheter is illustrated generally at 10. The illustrated apparatus includes a video camera 12 for direct visualization during use, a thumbwheel 14 for steering the device, a port 16 leading to a channel (not shown) for introduction of a sampling device, e.g., a brush, and an air valve 18 connected to an air source 20.

The apparatus may be an integrated device, or may comprise an existing scope, e.g., the DUR®-8 Ultra scope by Olympus, with a catheter add-on 22. FIG. 1B illustrates a cross-section of such catheter add-on through position 1 in FIG. 1A. Catheter add-on 22 includes an inflation lumen 24 for balloon 26 and an attachment lumen 28 complementary to scope 30.

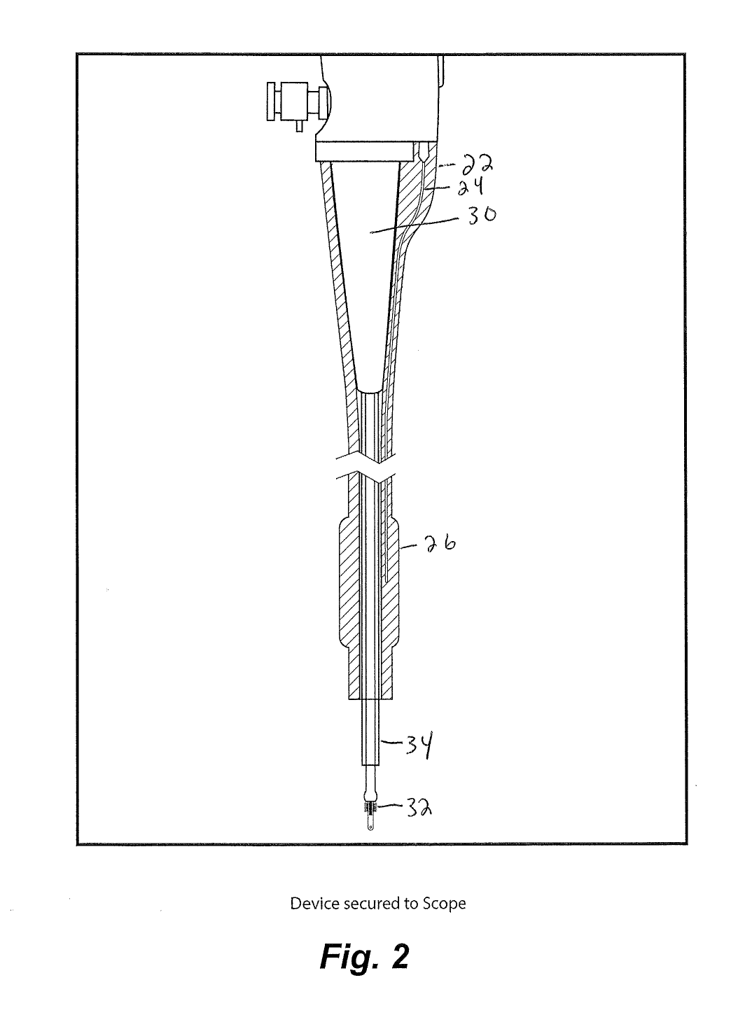

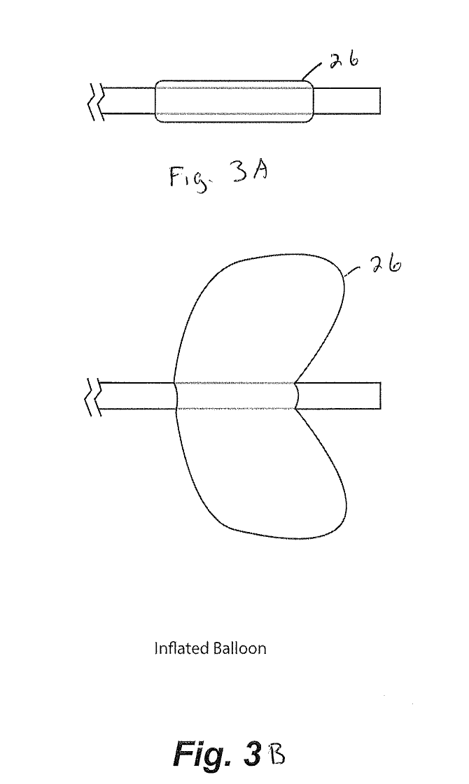

FIG. 2 illustrates the catheter add-on 22 in more detail, including the same identifiers. FIG. 2 also illustrates a cytological brush 32 extending from the channel 34 associated with the sampling port 16.

FIGS. 3A and 3B show the balloon 26 in non-inflated and inflated positions, respectively.

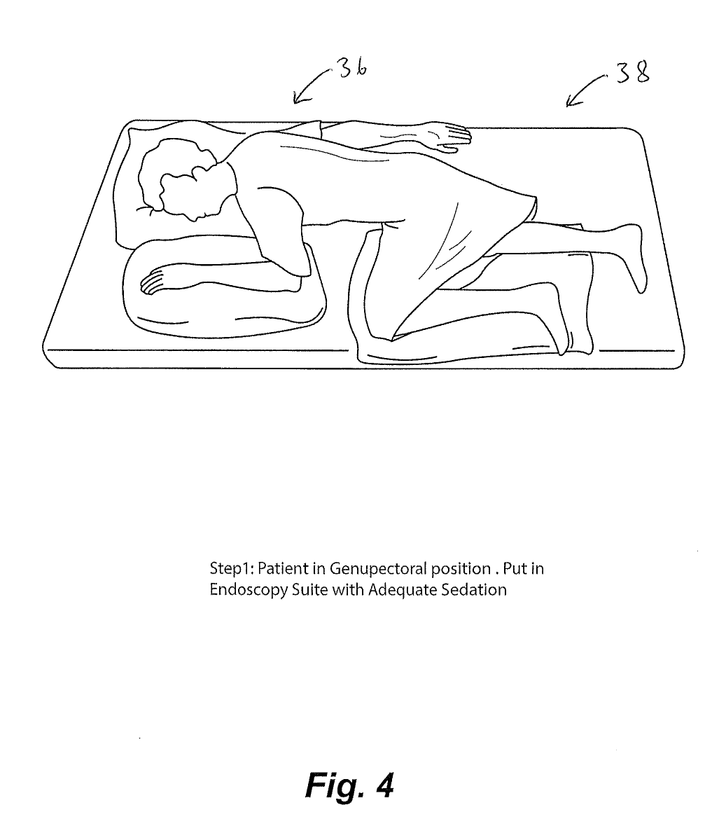

FIG. 4 illustrates the beginning of an exemplary procedure, including positioning of a patient, shown generally at 36 in a Genupectoral position in an endoscopy suite, shown generally at 38, with sedation.

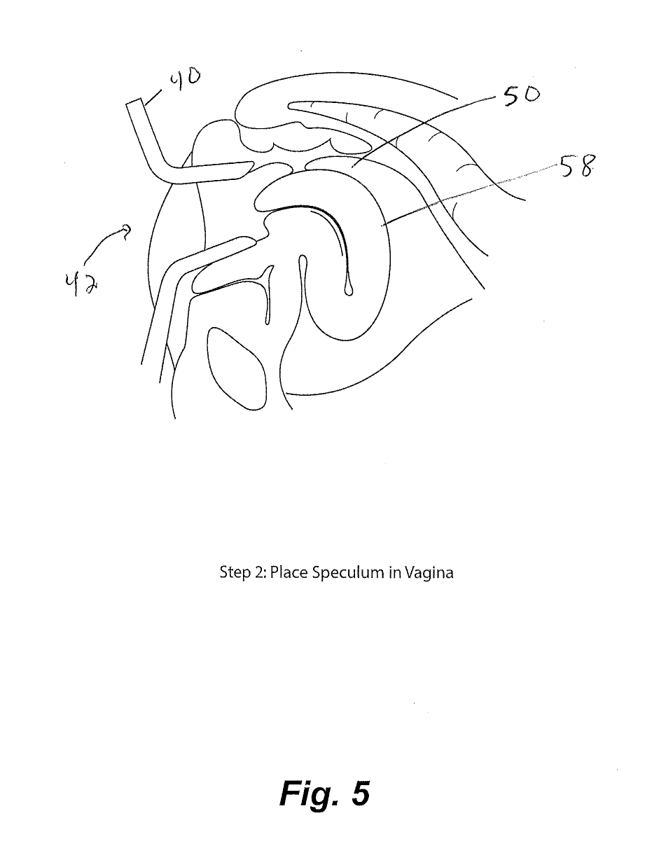

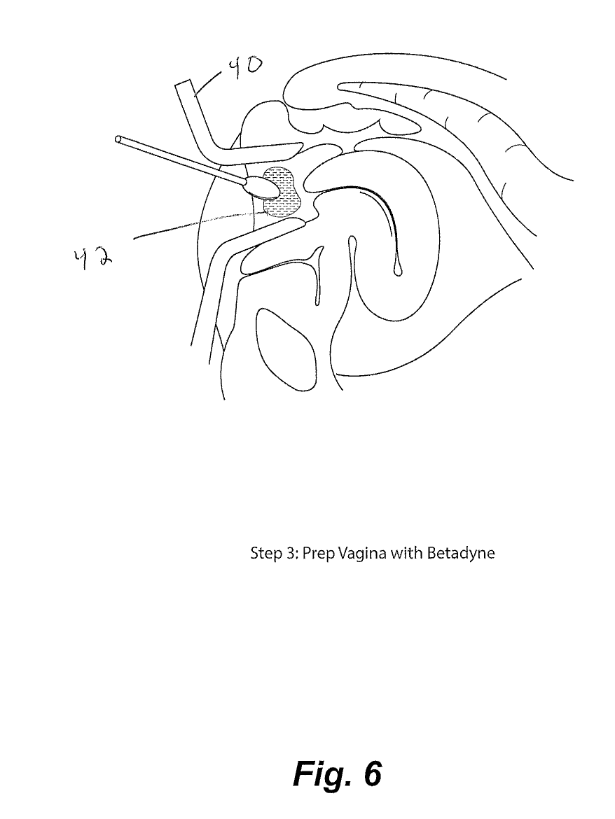

FIG. 5 illustrates use of a speculum (retractor) 40 in the vagina, shown generally at 42, to expose the posterior formix and visualize the cervix. FIG. 6 illustrates prepping of the vagina with Betadyne.

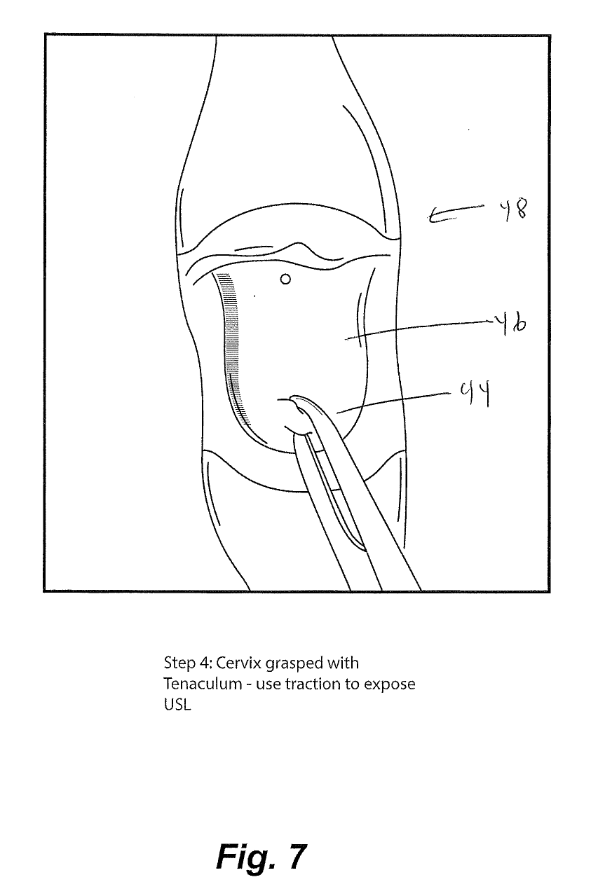

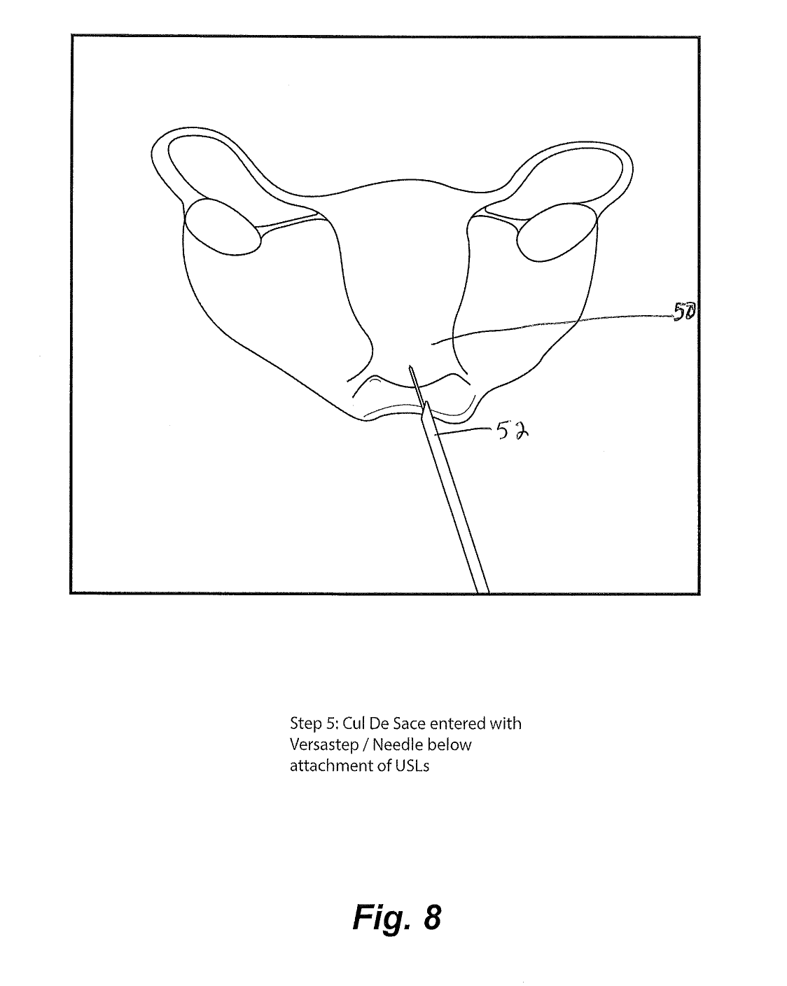

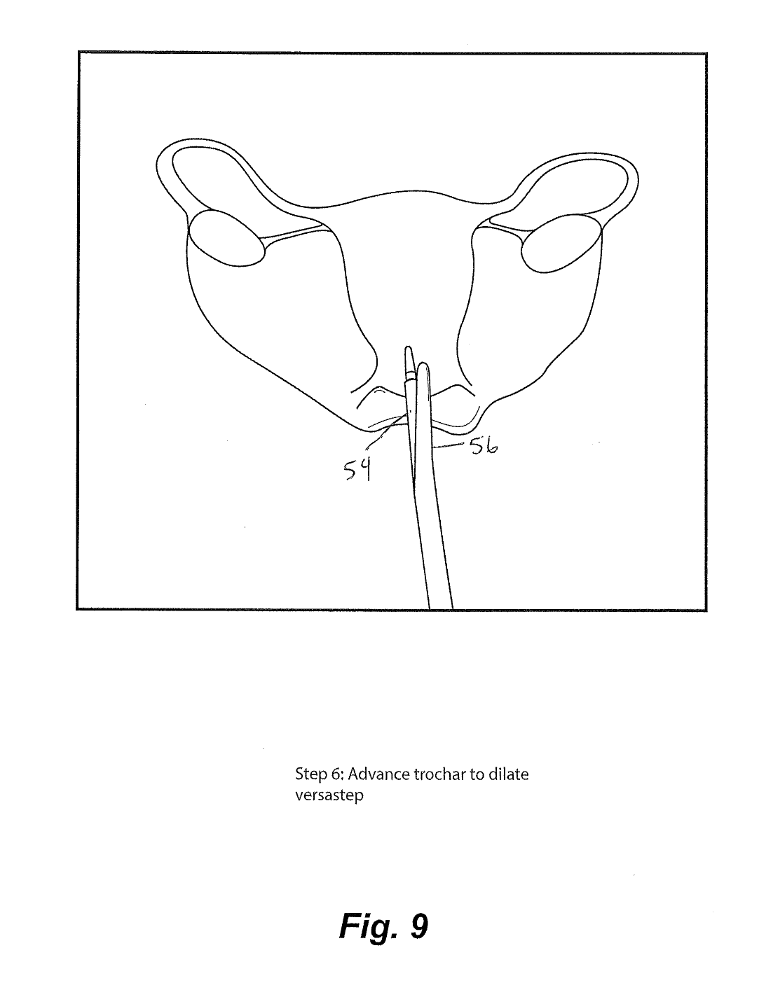

FIG. 7 illustrates grasping of the posterior lip 44 of the cervix 46 with a tenaculum, with traction applied to expose the uterosacral ligaments (USLs), shown generally at 48. As is shown in FIG. 8, the cul de sac 50 is entered between the USLs in the midline (1-2 centimeters away from the cervix) with a sheathed needle 52 (e.g., Versastep® with needle). Placement in the cul de sac may be confirmed by injecting 3-5 cc of fluid though the needle (or by aspirating the cul de sac fluid if the patient is not in the Genupectoral (knee-chest) position). The sheathed needle is removed, and as is illustrated in FIG. 9, a trocar 54 is advanced to dilate the sheath 56.

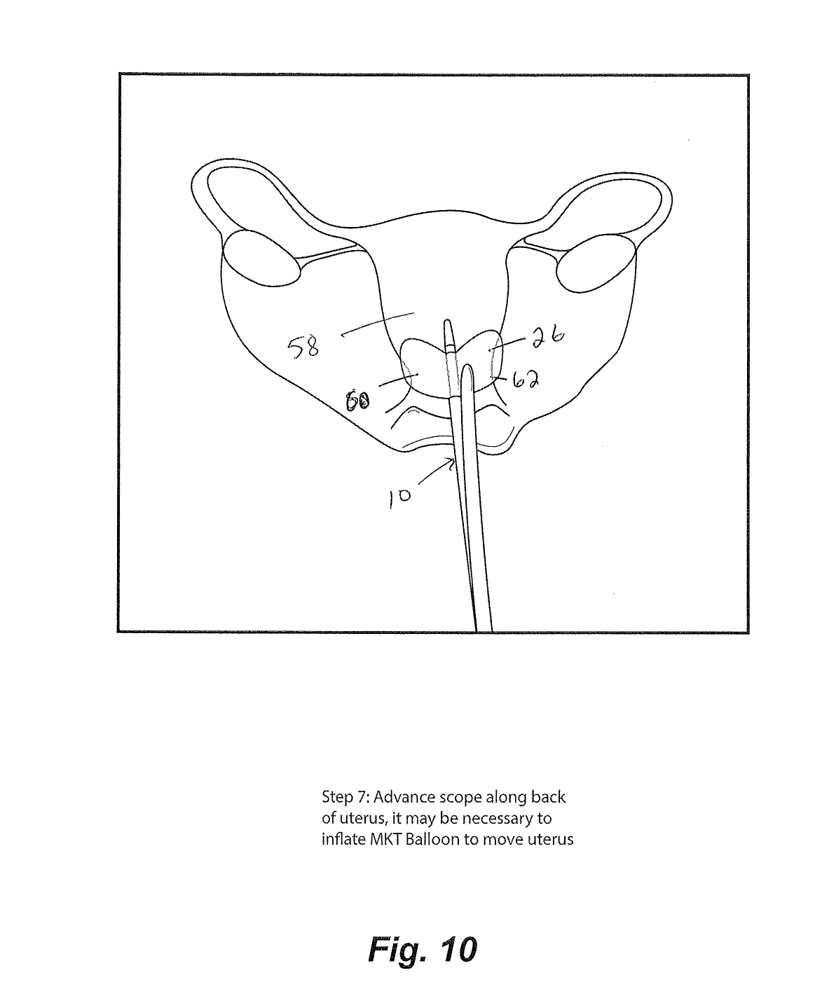

With regard to FIG. 10, the apparatus/catheter 10 is advanced along the back of the uterus 58, with optional inflation of the balloon 26 to move the uterus out of the way. As we have noted previously, the balloon is configured with two wings 60, 62 that are complementary to the shape of the uterus 58 (see also FIG. 5, which better shows the shape of the uterus 58 adjacent the cul de sac 50).

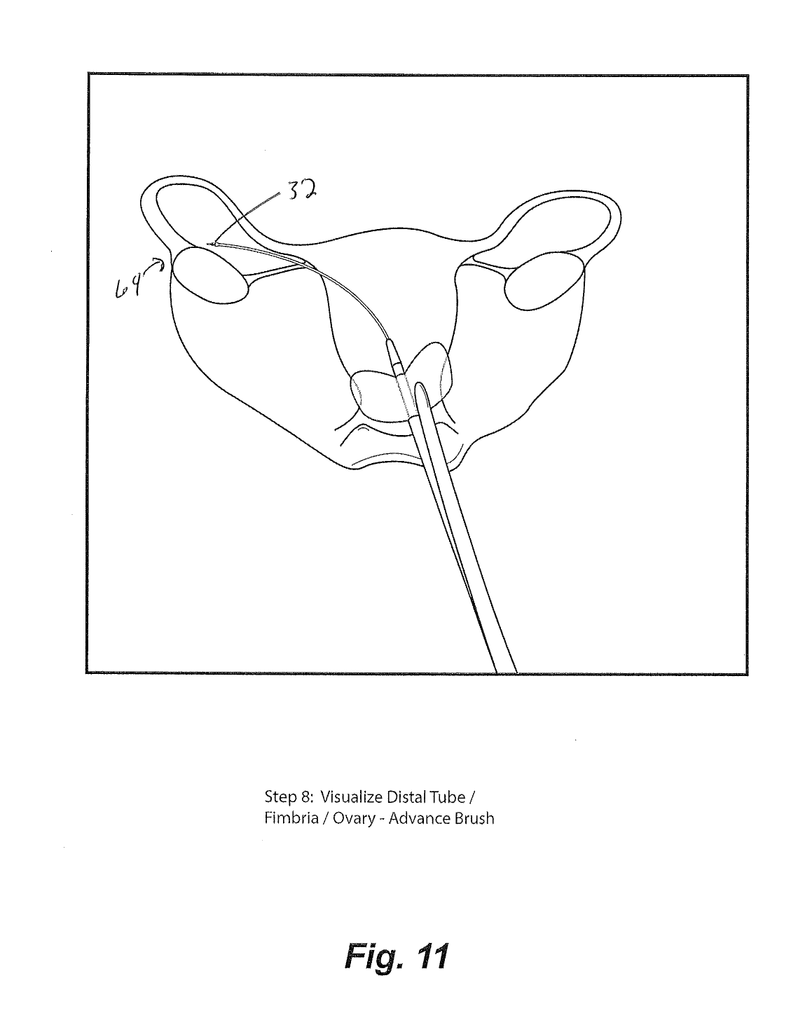

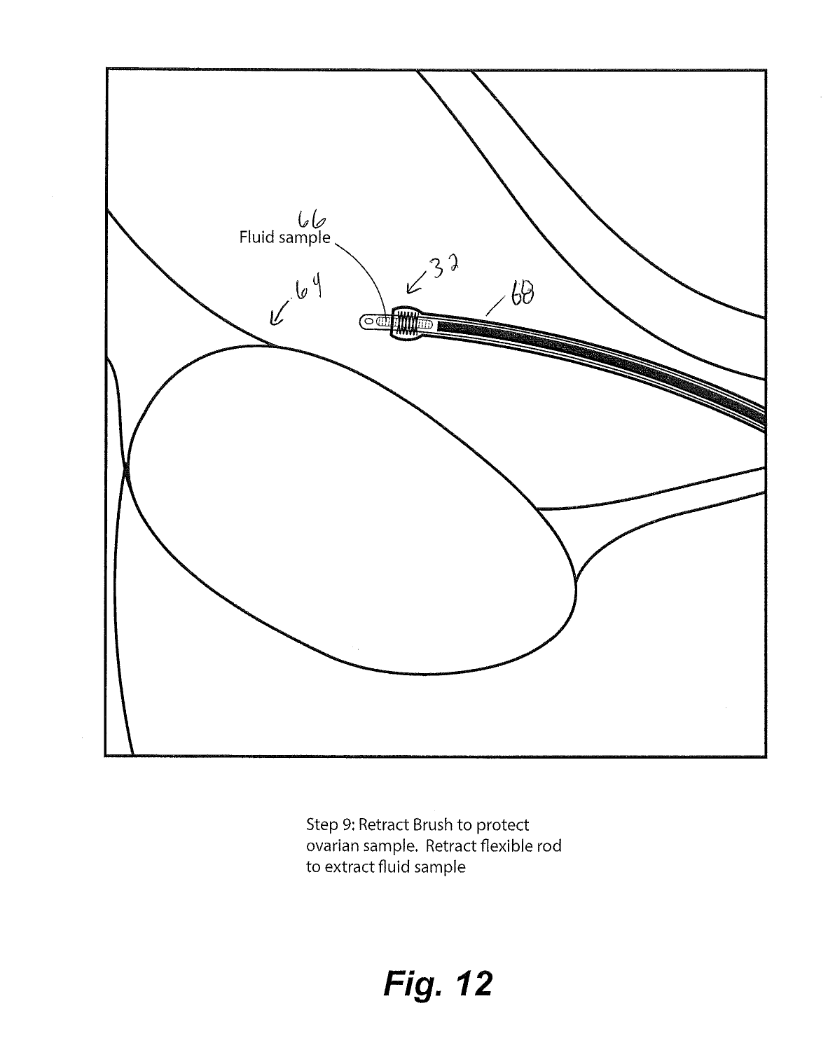

Referring now to FIG. 11, the scope is advanced to the uterine fundus, and then moved laterally to identify the fimbrial attachment, shown generally at 64, of the tube to the ovary. The brush 32 is advanced to sample the distal tube/proximal ovary at 64. FIG. 12 illustrates a close-up of the brush 32 with fluid sample 66 drawn from or proximate to the fimbrial attachment 64. The brush 32 is retracted into the flexible rod 68 to protect the ovarian sample 66, after which the flexible rod is retracted to extract the fluid sample.

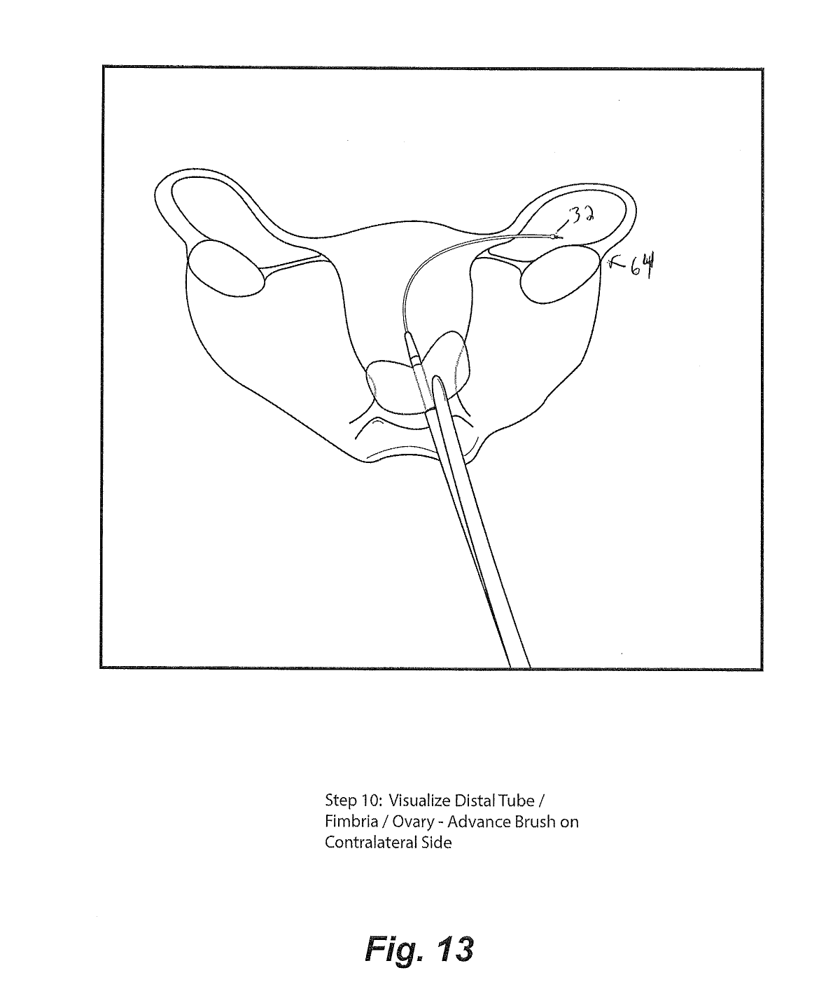

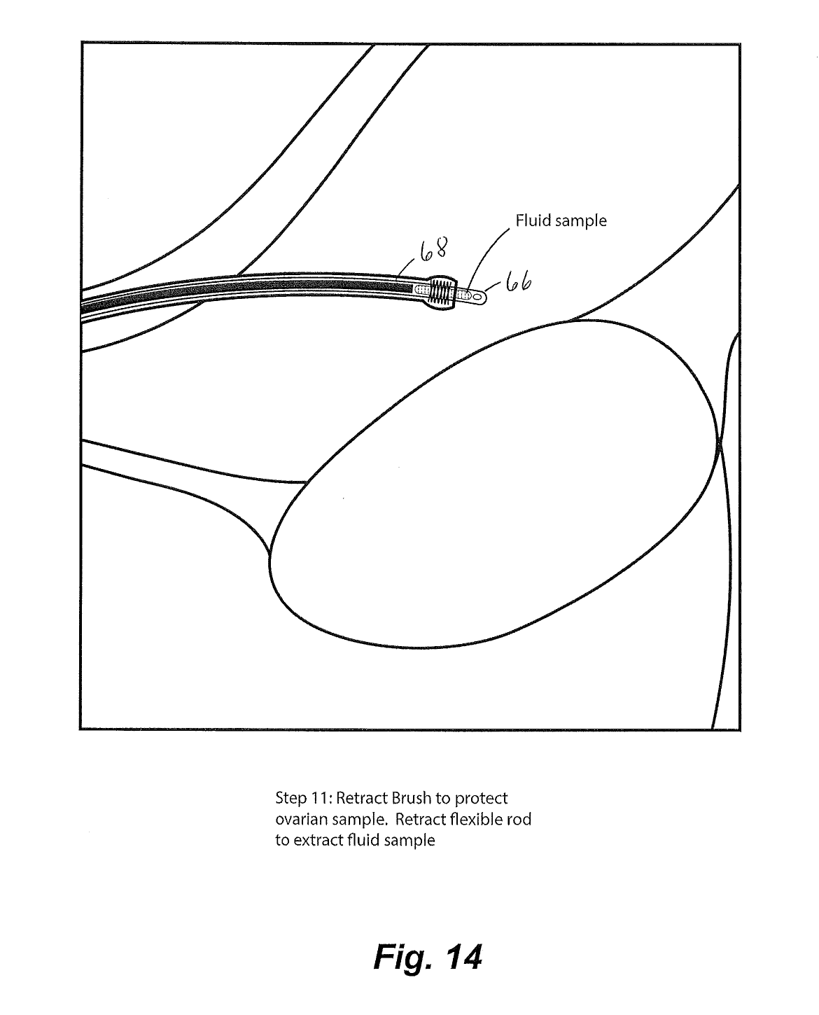

FIG. 13 shows sampling via a brush 32 on the contralateral side, after the scope is returned to the midline prior to sampling the opposite adnexa. FIG. 14 shows similar retracting of a fluid sample 66 into flexible rod 68.

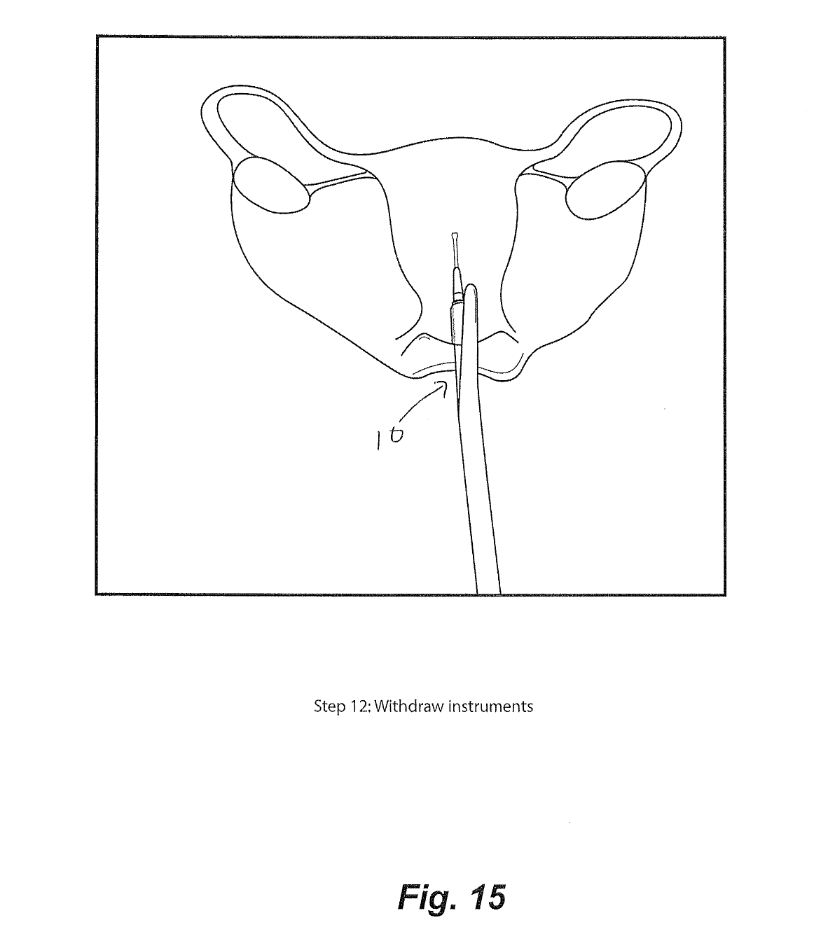

FIG. 15 shows removal of the apparatus 10.

Referring now to FIG. 16, an exemplary kit is illustrated generally at 70. This exemplary kit includes various exemplary items (it should be recognized that these items may be provided together, separately, or in various combinations). A first item, shown generally at 74, comprises a disposable grip 72 configured to attach to a handle of an existing scope, along with the catheter add-on 22.

A second item, shown generally at 76, comprises a sampling brush 32, a flexible rod 68 and a steering component 80, which allows the sampling brush to change directions. In the illustrated exemplary embodiment, the steering component is a twistable handle portion.

A third item, shown generally at 78, comprises a trocar for access to the cul de sac.

Various exemplary alternatives include different patient placement (e.g., the lithotomy position), use of aspiration for distal tube secretions and use of the procedure in various contexts (e.g., with or without colonoscopy, for screening of younger women with BRCA/high risk genetic mutations, office screening on awake patients, possibly under ultrasound guidance and screening of patients with known adnexal masses).

The present apparatus and method advantageously takes advantage of a culdoscopic approach in combination with sampling techniques from within the peritoneal cavity to identify ovarian cancer or precursor cells. The known art only uses imprint cytology, which is a smear of the ovary after ovarian removal (e.g., in the pathology lab), to assist in surgical decision making at the time of ovarian removal.

The presently described technique can also be provided as a brief screen of women concurrent with a colonoscopy. Women at risk for ovarian cancer are in the same age group as women who are traditional candidates for colonoscopy screening. In addition, the literature suggests a low complication rate for culdoscopy, the most common complication being bowel perforation; and women who present for colon cancer screening have already consented to a procedure with the same risk profile. Colonoscopy suites typically function with high volume and quick room turnover, and the present method could reasonably be performed within 30 minutes.

Additionally, patients having screening colonoscopy have completed a bowel prep for the colonoscopy, which would reduce the risk of infection should an inadvertent bowel puncture occur (however, this should not be common where the presently described procedure is performed under direct visualization).

Patients identified with cancer would be better able to be counseled preoperatively and indeed may opt to schedule their procedure at a different time or facility so that a gynecologic-oncologist would be readily available. Women who have benign results may also be able to observe pelvic cysts. Additionally, in the case of women with genetic risk for cancer, reassuring results may assist in the decision regarding timing of risk reduction surgery (e.g., many women want to complete their families or avoid menopausal symptoms until later ages).

The presently described procedure may also be useful in pre-operative planning for patients why by be scheduling procedures at facilities where a gynecological oncologist may not be readily available.

Finally, the present apparatus and method provide for direct sampling from the ovary/fallopian tube complex without removal of potentially normal organs.

### Example 1

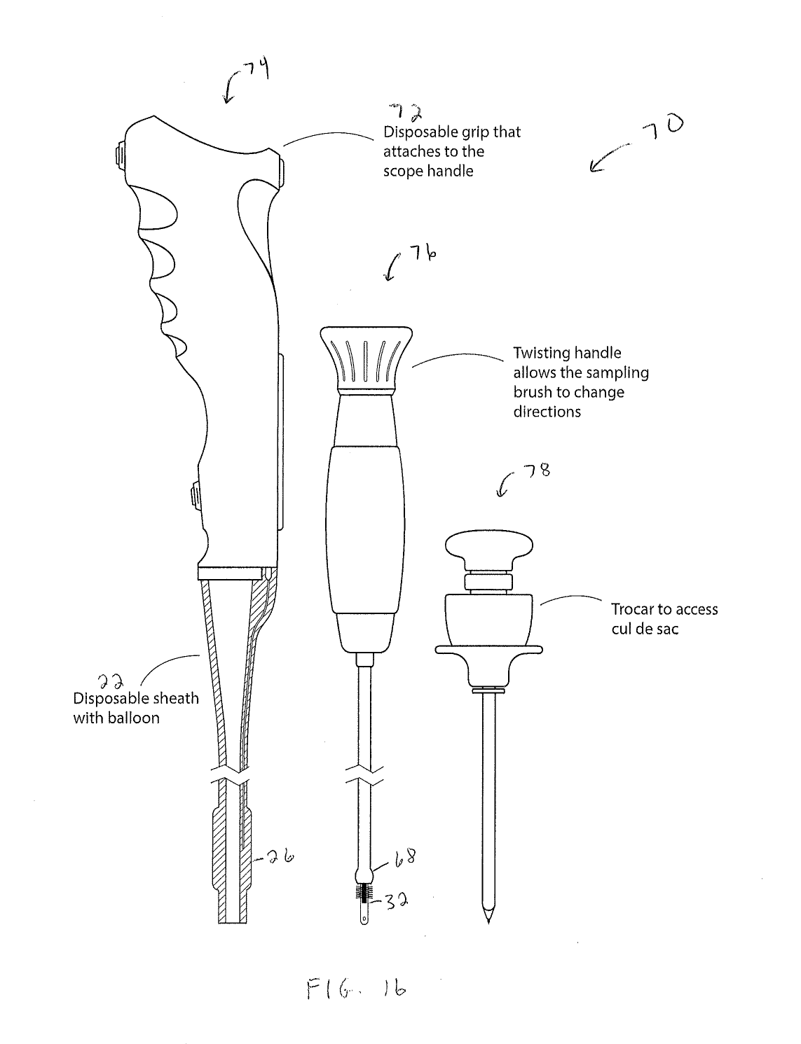

An exemplary procedure is described immediately below with reference to FIGS. 17-24:

1. Insert catheter assembly into 5 mm port in the uteroscope;

2. Insert sampling device into scope working channel (brush end first, the handle is connected on the other end);

3. Handle should be about ½ inch from touching the working channel;

4. Insert scope into scope lumen;

5. Inflate the balloon by attaching a non-depressed standard 30 mL syringe to the stopcock. Once desired inflation is complete close stopcock and disconnect syringe (this is optional but it allows for more room if you remove the syringe.);

6. Position the end of the uteroscope at the end of one fallopian tube;

7. Connect depressed 3 mL syringe to luer lock and remove approximately 1 mL of fluid. If required, flush the internal with saline to obtain necessary volumes. Place fluid in standard cup to send to lab;

8. Move the end of the sampling device to the surface of the ovaries. Click the button which will extend the brush. Once cell collection is completed, press the button on the handle body to retract the brush into its protective sheath;

9. Remove sampling device from the uteroscope. Cut the brush and drop into pouch for testing in the lab. Dispose of remainder of sampling tube; and

10. Repeat steps 8-9 for the other ovary and fallopian tube.

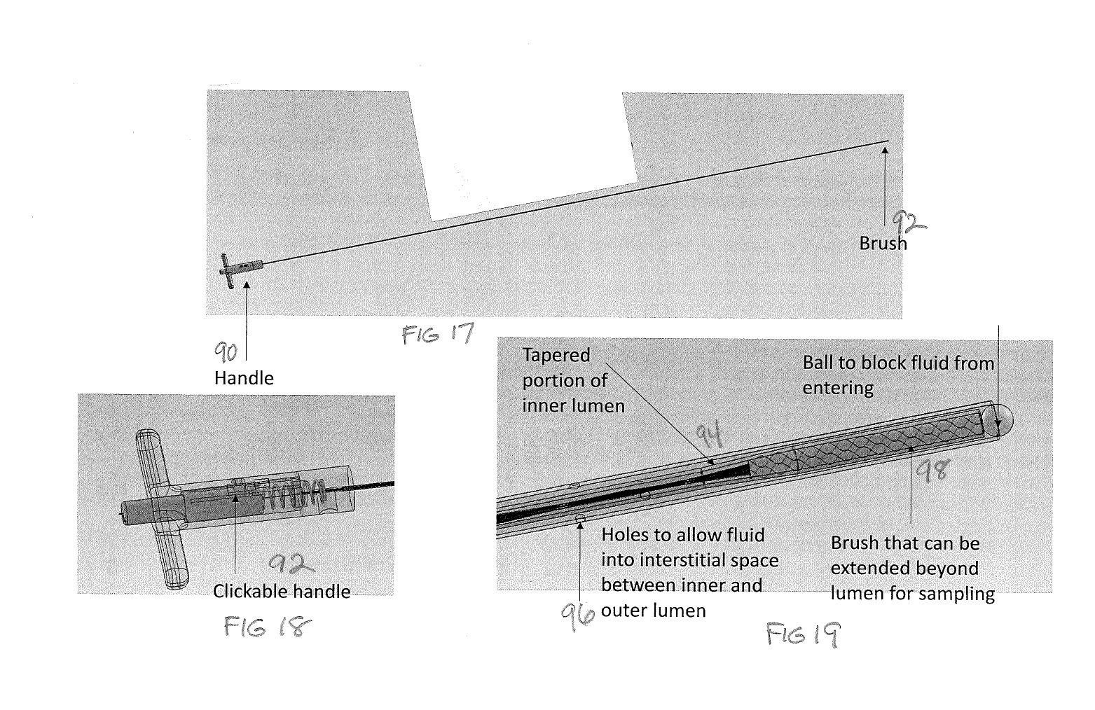

FIG. 17 illustrates a handle 90 attached to a brush 92, with a length sufficient between the two to go through a uteroscope. A clickable mechanism within the handle is illustrated at 92 in FIG. 18. FIG. 19 shows a tapered portion of the inner lumen at 94, with holes 96 to allow fluid into the interstitial space between the inner and outer lumen. A brush 98 can be extended beyond the lumen for sampling,

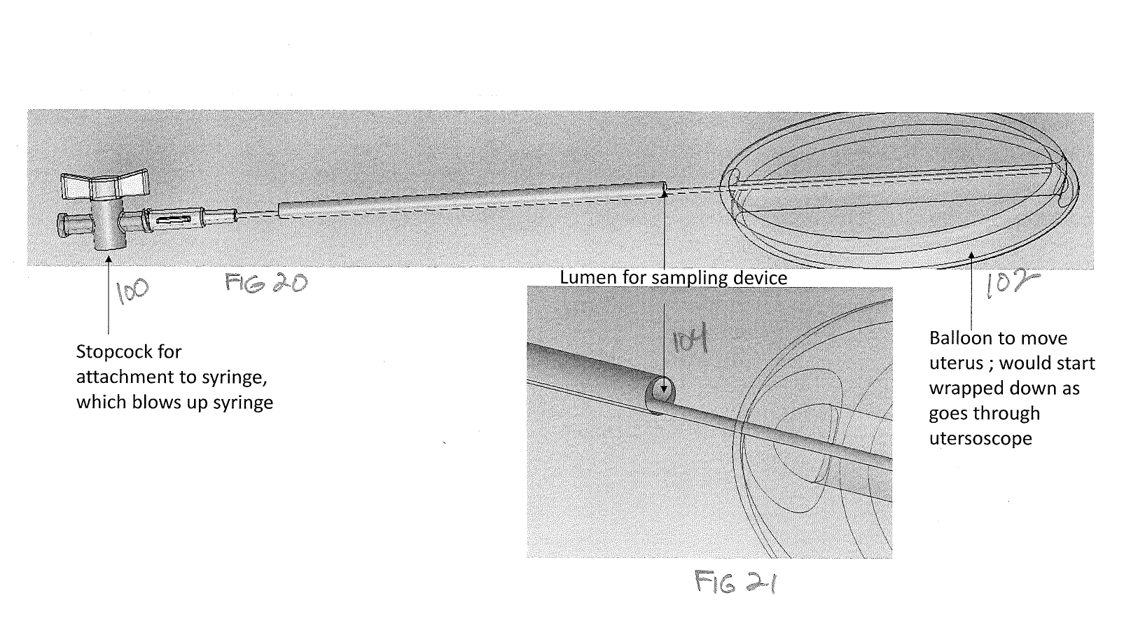

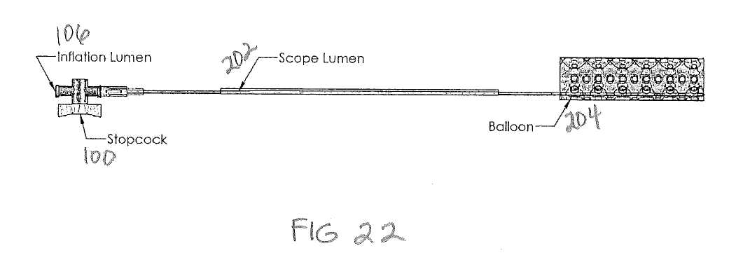

FIGS. 20 and 21 illustrate a stopcock 100 for attachment to a syringe, which blows up the balloon, a lumen 102 for the sampling device; and a balloon 104 to move the uterus (which balloon may start wrapped down as it goes through the uteroscope.

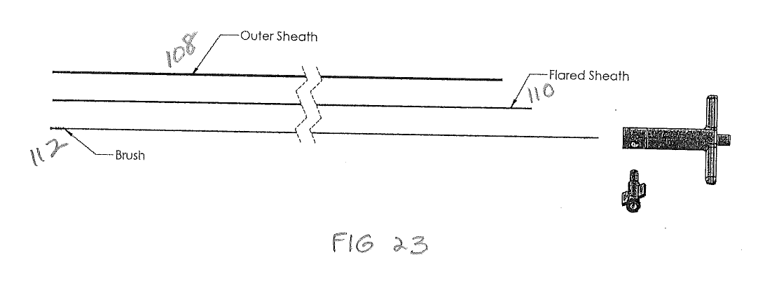

FIG. 22 illustrates an exemplary device, including inflation lumen 106, a stopcock 100; a scope lumen 102 and a balloon 104.

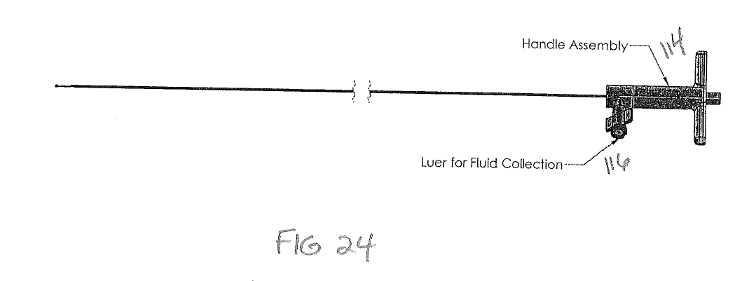

FIG. 23 further illustrates an outer sheath 108; a flared sheath 110; and a brush 112.

FIG. 24 illustrates a handle assembly 114 and a Luer 116 for fluid connection.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A method for sampling the distal tube, fimbria and/or ovary via catheter accessing the peritoneal cavity through the cul de sac, comprising:
piercing the cul de sac through the vaginal wall;
advancing a catheter through the cul de sac and inflating a balloon operatively associated with the catheter to lift the uterus;
continuing advance the catheter into the peritoneal cavity to a position proximate to a distal tube, fimbria or ovary;
sampling material on or adjacent to the distal tube, fimbria or ovary; and
deflating the balloon and withdrawing the catheter with the sampled material.

2. A method in accordance with claim 1, wherein sampling is performed with a brush that extends from and retracts into said catheter.

3. A method in accordance with claim 1, wherein said catheter additionally samples on a contralateral side.

4. A method in accordance with claim 1, wherein said catheter comprises a scope configured to visualize a target.

5. A method in accordance with claim 1, wherein said catheter includes a steerable distal portion.

6. A method in accordance with claim 1, wherein said balloon is configured with two wings complementary to the natural shape of the uterus.

7. A method in accordance with claim 1, wherein access is facilitated with the use of a vaginal speculum.

8. A method in accordance with claim 1, wherein access is facilitated by a tenaculum to expose the access point.

9. A method in accordance with claim 1, wherein access is provided by a sheathed needle, followed by a trocar to dilate said sheath.

10. A method in accordance with claim 1, wherein sampling material comprises uses one or more of a retractable brush, a stationary brush, a needle, a cutting device, a suction device, or a powered removal device.

11. A method in accordance with claim 10, wherein said powered removal device comprises a laser cutting device or an electric knife.

12. A method in accordance with claim 10, wherein said cutting device comprises scissors or a grader.

13. A system for sampling the distal tube, fimbria and/or ovary via catheter accessing the peritoneal cavity through the cul de sac, comprising:
a scope;
a scope add-on, configured with an inflatable balloon, the balloon configured an inflatable contour complementary to the natural shape of the uterus; and
a brush channel on one of the scope or the scope add-on, the brush channel configured to accept a movable brush configured to sample material at a site of interest.

14. A system for sampling the distal tube, fimbria and/or ovary via catheter accessing the peritoneal cavity, comprising:
a scope;
a scope add-on, configured with an inflatable balloon, the balloon configured an inflatable contour complementary to the natural shape of the uterus; and
a sampling device on one of the scope or the scope add-on.

15. A system in accordance with claim 14, wherein said balloon is configured with two wings that are complementary to the natural shape of the uterus.

16. A medical device for sampling the distal tube, fimbria and/or ovary via catheter accessing the peritoneal cavity through the cul de sac, comprising:
a scope, configured with an inflatable balloon, the balloon configured an inflatable contour complementary to the natural shape of the uterus; and
a brush channel, the brush channel configured to accept a movable brush configured to sample material at a site of interest.

17. A device in accordance with claim 16, wherein said balloon is configured with two wings that are complementary to the natural shape of the uterus.

18. A method for sampling the distal tube, fimbria and/or ovary through the cul de sac, comprising:
piercing the cul de sac through the vaginal wall;
advancing a catheter through the cul de sac; and
sampling material on or adjacent to the distal tube, fimbria or ovary.

19. A method in accordance with claim 18, wherein sampling is performed with a brush that extends from and retracts into said catheter.

20. A method in accordance with claim 18, wherein said catheter additionally samples on a contralateral side.

21. A method in accordance with claim 18, wherein said catheter comprises a scope configured to visualize a target.

22. A method in accordance with claim 18, wherein said catheter includes a steerable distal portion.

23. A method in accordance with claim 18, wherein said catheter includes a balloon that is configured with two wings complementary to the natural shape of the uterus.

24. A method in accordance with claim 18, wherein access to the cul de sac is facilitated with the use of a vaginal speculum.

25. A method in accordance with claim 18, wherein access to the cul de sac is facilitated by a tenaculum to expose the access point.

26. A method in accordance with claim 18, wherein access to the cul de sac is provided by a sheathed needle, followed by a trocar to dilate said sheath.

27. A method in accordance with claim 18, wherein sampling material comprises uses one or more of a retractable brush, a stationary brush, a needle, a cutting device, a suction device, or a powered removal device.

28. A method in accordance with claim 27, wherein said powered removal device comprises a laser cutting device or an electric knife.

29. A method in accordance with claim 27, wherein said cutting device comprises scissors or a grader.

30. A method for sampling the distal tube, fimbria and/or ovary, comprising:
advancing a device into the peritoneal cavity; and
sampling material on or adjacent to the distal tube, fimbria or ovary.

31. A method in accordance with claim 30, wherein said device is a scope and/or catheter.

32. A method in accordance with claim 30, wherein the device is configured to deposit materials on or near the distal tube, fimbria or ovary.

33. A method in accordance with claim 30, wherein a balloon is provided configured with an inflatable contour complementary to the natural shape of the uterus, and wherein such contour is circular or cylindrical so as to allow a sampling brush to extend through the center or portion of said balloon.

34. A method for delivering medicine to the distal tube, fimbria and/or ovary, comprising:
advancing a device into the peritoneal cavity; and
depositing material on or adjacent to the distal tube, fimbria or ovary.

35. A method for delivering medicine to the distal tube, fimbria and/or ovary through the cul de sac, comprising:
piercing the cul de sac through the vaginal wall;
advancing a catheter through the cul de sac; and
depositing medicine on or adjacent to the distal tube, fimbria or ovary.
